(19) United States
(12) Patent Application Publication
(10) Pub. No.: US 2022/0072324 A1
(43) Pub. Date: Mar. 10, 2022

(54) METHOD AND SYSTEM FOR APPLYING PULSED ELECTRIC FIELDS WITH HIGH UNIFORMITY USING MAGNETIC CORES

(71) Applicants: Board of Supervisors of Louisiana State University and Agricultural and Mechanical College, Baton Rouge, LA (US); The Phantom Laboratory, Incorporated, Salem, NY (US)

(72) Inventors: Dennis J. Paul, New Orleans, LA (US); Harry J. Gould, New Orleans, LA (US); Richard Philip Mallozzi, Middleton, MA (US)

(73) Assignees: Board of Supervisors of Louisiana State University and Agricultural and Mechanical College, Baton Rouge, LA (US); The Phantom Laboratory, Incorporated, Salem, NY (US)

(21) Appl. No.: 17/299,838

(22) PCT Filed: Dec. 2, 2019
(86) PCT No.: PCT/US2019/063984

§ 371 Date: Jun. 4, 2021

Related U.S. Application Data
(60) Provisional application No. 62/776,105, filed on Dec. 6, 2018.
(60) Provisional application No. 62/942,348, filed on Dec. 2, 2019.

(51) Int. Cl. A61N 1/40; H01F 7/20; H01F 7/06

(52) U.S. Cl. CPC A61N 1/40; H01F 7/064; H01F 7/20

(57) ABSTRACT

A device, system and method for creating pulsed electric fields with good uniformity are provided. The electric fields occupy a large volume, suitable for placing a human or animal patient. A device for generating the electric fields is provided, which comprises one or more toroidally or cylindrically shaped magnetic core(s) wound with electrical wires, which, when pulsed with electrical currents, generate an electric field of high uniformity in the interior region of the toroidal or cylindrical cores. These electric field pulses, when used in conjunction with pharmacological agent, destroy cancer cells through a process called Targeted Osmotic Lysis.

### RELATED APPLICATION

This application is a § 371 National Stage Application of PCT/US2019/063984, filed Dec. 2, 2019, which claims priority benefit to U.S. Provisional Application No. 62/776,105, filed Dec. 6, 2018, and U.S. Provisional Application No. 62/942,348, filed Dec. 2, 2019, both of which are fully incorporated herein by reference for all purposes.

### FIELD

The present invention relates to the field of medical device and medical treatment of diseases and disorders. More specifically, the invention concerns a method and system for applying pulsed electric fields with high uniformity using toroidal or cylindrical magnetic cores for medical applications.

### BACKGROUND ART

Pulsed electric field treatment is now widely used in diverse biological and medical applications: gene delivery, electrochemotherapy, and cancer therapy. One advantage of pulsed electric field treatment, is the ability to destroy tissues or tumors in a nonthermal manner. Consequently, pulsed electric field treatment makes it possible to preserve sensitive tissues intact, such as blood vessels and axons. Furthermore, this non-invasive technique allows the possibility of regeneration with healthy cells and tissues in the treatment region without leaving a scar and unlike stimulation with electric current, the delivery of the stimulus is significantly more uniform.

Conventional appliance consists of three parts: pulse generator, electrodes, and connection links between them. The pulse generator produces square wave pulses at regular intervals. Amplitude, pulse width, period, and phase delay are the primary parameters to determine the shape of the output waveform. Electric field strength, depending on the amplitude of the pulse and the distance between the electrodes, is often crucial for completed treatment effect. When electrodes are unsuitable, the strength in a certain target area is insufficient, resulting in incomplete treatment effects.

The electric field is generated by equipment similar to that used in radar. This has some consequences for cost and availability. The most typical equipment generates a short square wave and reverses polarity, in part to avoid erosion of electrodes. However, a bipolar generator costs about twice as much as a monopolar one. Other wave forms include exponential decay and sinusoidal. The sinusoidal form is somewhat easier to generate, as it uses equipment similar to common radio equipment, but it reaches its peak power only for an instant and so delivers less energy per cycle above the critical field strength than does a square wave.

Two alternatives are apparent for generating such electric field pulses. The first is to use a solenoidal arrangement, such as a large coil of wires that generates a uniform magnetic field on the interior. The patient would be placed inside the solenoid. However, this technique has major disadvantages. The first is that the generated electric field is zero along the axis of such a solenoid, and increases linearly with the distance from the center axis. Thus the electric field not only disappears in the most critical region, but is highly non-uniform everywhere. This non-uniformity is, in fact, an inherent limitation to any technique in which the patient is placed within the region of the ramped magnetic field. The electric field within a region of changing magnetic field will necessarily be non-uniform due to the physics of electromagnetic fields. The second disadvantage of such a technique is the amount of heat that such a coil would generate. If such a coil is designed that is large enough to fit a human patient or moderately-sized animal within the interior, the coil would generate extremely large quantities of heat that would not only have to be removed from the device, but impose large HVAC requirements on the building in which the device was applied. For a device scaled up for application to a human or large-animal size, peak thermal power in the range of 50-400 kilowatts would be generated. Such power requirements present a large challenge for building facilities.

A second alternative for generating the electric fields is to use an arrangement of charged objects, such as conducting plates, to generate the electric field. A disadvantage of this technique is that the most practical implementations involve applying the electric field in a manner such that the field points align perpendicular to the surface of the patient, thereby causing a large reduction in the electric field inside the patient due to dielectric polarization of the water molecules within the tissue. Furthermore, the actual field will be very sensitive to the percentage of the space between the plates that is filled by the patient. An additional disadvantage of the technique is the sensitivity to the geometry of the patient. A patient with larger girth would require different applied voltages. Different regions of the body that have different size, such as arms and legs compared to torso, would receive very different electric field dose. The plates could be placed directly in contact with the patient, but unless the plates are flexible, they will only contact a portion of the patient's skin. In this way, a significant amount of energy is required to overcome the skin's impedance and that electrical stimulation is not comfortable and often painful. Such limitations are practical in nature, and it is believed that the method disclosed here offers numerous advantages to such an approach.

Although advances have been made recently in the use of electric pulses to induce cell death, there still exists a need in the art for improved methods for destroying diseased or disordered tissues, such as tumor tissues, without damaging normal tissues. Especially there is a need for methods and systems of generating pulsed electric fields in large volumes with high uniformity.

### SUMMARY OF THE INVENTION

In view of the foregoing, the object of the present invention is to address the need for generating pulsed electric fields in large volumes with high uniformity for medical applications. Embodiments of the present invention pertain to a method and system for creating pulsed electric fields with amplitudes in the range of 0-100 Volts/meter in empty space, with durations in the range of 1-50 milliseconds each. The electric fields occupy a large volume, suitable for placing a human or animal patient. The electric field has good uniformity.

One embodiment provides a device for generating pulsed electric fields, which comprises a multiplicity of toroidally-shaped magnetic cores wound with electrical wires. Another embodiment provides a device for generating the electric fields, which comprises a multiplicity of magnetic cores shaped in a cylindrical geometry, such that the magnetic material occupies an annular region around the axis of the cylinder, and the central region of the cylinder is empty. Various designs include cores that resemble long cylinders, and short cores that resemble a toroid geometry. The magnetic cores are wound with electrical wires, which, when pulsed with electrical currents, generate an electric field of high uniformity in the interior region of the toroidal or cylindrical cores. These electric field pulses, when used in conjunction with pharmacological agent, destroy cancer cells through a process called Targeted Osmotic Lysis (TOL). See U.S. Pat. No. 8,921,320.

The magnetic cores are constructed out of material with high magnetic permeability. When current is applied to the electrical windings, a large magnetic field is produced inside the magnetic material. This field is most uniform along the axis of the toroidal or cylindrical cores, near the center of the device. Application of a time-dependent current leads to a changing magnetic field inside the core, which in turn creates an electrical field outside the core material that can be used for the therapy. The amplitude, duration, and temporal spacing of the electric field pulses can be controlled by controlling the voltage and current applied to the electrical windings.

The important properties of the electric field produced are high uniformity, and a direction that points along the long axis of a human subject, or many animal subjects. Another important aspect of the device is that it produces the electric fields with very low power generated, leading to low-cost driving electronics, low electrical requirements for a facility, and no impact on the HVAC systems of a clinical facility.

The toroidal or cylindrical cores are arranged coaxially, at separations that optimize the uniformity of the electric field produced. The electric field produced points along the axial direction of the toroidal or cylindrical cores. The patient is placed in the interior region along the axis of the toroidal or cylindrical cores.

The system consists of the toroidal or cylindrical device, connected to a set of driving electronics that allow the user to control the amplitude, duration, and spacing of the electrical field pulses. The electronics consist of components to generate pulsed voltage or current waveforms, components to amplify and filter the output of the waveforms, and a microprocessor that presents a user interface for controlling the output.

In particular, one embodiment provides a device for generating pulsed electric fields, comprising:
one or more toroid or cylinder structure(s),
a plurality of conducting windings wrapped around each toroid or cylinder structure, and
a plurality of wires that supply electrical current to the conducting windings.

According to one embodiment, the toroid structure is made of magnetic material with high relative magnetic permeability in the range of 1,000 to 40,000. The magnetic material with a magnetic permeability of 40,000 or higher can be used in this application. Some examples of suitable magnetic materials include, but not limited to, silicon steel, powdered iron, nickel-iron alloys, ferrite ceramics, nanocrystalline alloys of iron, boron, and silicon. The conducting winding is made of copper wire in the range of 10-28 AWG and is wound into a coil arrangement on the toroid structure with typically 1 to 200 turns. Other examples of suitable material for the conducting winding include, but not limited to, aluminum, silver, tin, galvanized steel, phosphor bronze, lead, and gold. The magnetic field inside the magnetic material is generated when an electrical current is ramped through the conducting windings.

According to one embodiment, a plurality of the toroid structures is aligned coaxially and separated by a distance equal to the radius of the toroid structure. In at least another embodiment, three toroid structures are arranged axially, with one toroid structure of larger radius situated halfway between two identical toroid structures.

According to another embodiment, the cylinder structure is made of magnetic material with high relative magnetic permeability, typically higher than 1,000. Some examples of suitable magnetic materials include, but not limited to, silicon steel, powdered iron, nickel-iron alloys, ferrite ceramics, nanocrystalline alloys of iron, boron, and silicon. The conducting winding is made of copper wire in the range of 10-28 AWG and is wound into a coil arrangement on the cylinder structure with typically 1 to 200 turns. Other examples of suitable material for the conducting winding include, but not limited to, aluminum, silver, tin, galvanized steel, phosphor bronze, lead, and gold. The magnetic field inside the magnetic material is generated when an electrical current is ramped through the conducting windings.

According to another embodiment, a plurality of the cylinder structures is aligned coaxially. The spacing between the cylinder structures and the current run through the coils surrounding each cylinder structure is adjusted to provide optimal electric field strength and uniformity, as well as to have desirable electrical properties such as inductance and resistance.

According to at least one embodiment, a device for generating pulsed electric fields further comprises an additional coil winding used for sensing the electric field generated within the device. The additional coil winding comprises a loop of wire encircling the cross section of toroidal or cylindrical structure with at least one turn, or more for a higher sensitivity to the electric field, wherein the loop of wire induces a voltage which is proportional in magnitude to the rate of change of magnetic flux through the cross section of toroidal or cylindrical structure.

Another embodiment relates to a system for therapeutic treatments involving electric fields, comprising:
a toroid or cylinder device for generating pulsed electric fields,
a driving and sensing circuitry,
a plurality of cables connecting the device to the driving and sensing circuitry, and
a microprocessor providing a user interface for operating the device and the driving and sensing circuitry.

According to one embodiment, the magnetic toroid or cylinder device comprises one or more toroid or cylinder structure(s), conducting windings wrapped around the toroid or cylinder structures, and wires that supply electrical current to the conducting windings. The pulsed electric fields are used in the application of targeted osmotic lysis for treating cancers when combined with pharmacological agents.

A further embodiment relates to a method for therapeutic treatments involving electric fields, comprising generating pulsed electric fields by a magnetic toroid or cylinder device, wherein the magnetic toroid or cylinder device comprises one or more magnetic toroid or cylinder structure(s), conducting windings wrapped around the toroid structure, and wires that supply electrical current to the conducting windings. This method can be used in the application of targeted osmotic lysis for treating cancers when combined with pharmacological agents.

### BRIEF DESCRIPTION OF THE DRAWINGS

The invention will be described in more detail below on the basis of one or more drawings, which illustrates exemplary embodiments.

FIG. 1. Single toroid with winding depicted. The toroid consists of a high-magnetic-permeability magnetic material, an example of which is silicon steel.

FIG. 2. Single cylinder with winding depicted. The cylinder consists of a high-magnetic-permeability magnetic material, an example of which is silicon steel.

FIG. 3. A toroid or cylinder structure having a magnetic structure that is closed except for a gap wide enough to place the relevant anatomy.

FIG. 4. Two toroidal cores arranged in a manner to create electric field in the empty region on the interior of the toroidal cores.

FIG. 5. A plurality of cylinders arranged coaxially to provide an extended region of electric field exposure

FIG. 6. A loop of wire that can encircle the toroid cross section once, or more than once for a higher sensitivity to the electric field.

FIG. 7. Therapeutic system comprising toroidal or cylindrical cores in an enclosure and connected to a control system for application of therapy involving electric fields.

FIG. 8. A typical pulse train associated with the TOL application.

FIG. 9. Mice treated with TOL reached humane endpoint euthanasia criteria significantly longer than mice in the control groups. Arrows indicate days of treatment.

### DETAILED DESCRIPTION OF THE EMBODIMENTS

It should be understood that this invention is not limited to the particular methodology, protocols, and systems, etc., described herein and as such may vary. The terminology used herein is for the purpose of describing particular embodiments only and is not intended to limit the scope of the present invention, which is defined solely by the claims.

As used in the specification and appended claims, unless specified to the contrary, the following terms have the meaning indicated below.

“Relative permeability” refers to the ratio of the permeability of a specific medium to the permeability of free space.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The following description and the drawings sufficiently illustrate specific embodiments to enable those skilled in the art to practice them. Other embodiments may incorporate structural, logical, electrical, process, and other changes. Portions and features of some embodiments may be included in, or substituted for, those of other embodiments.

This invention addresses a need to create pulsed electric fields in large (human-body-sized) volumes. This need arises within the application of Targeted Osmotic Lysis (TOL), which uses such electric field pulses to stimulate sodium channels in the cell membrane of cancer cells to open. See U.S. Pat. No. 8,921,320. It is desirable to have the electric field highly uniform so that the associated therapeutic effect will be uniform.

The technique involves using a ramped magnetic field that is confined to a high-magnetic-permeability material, such as a transformer core. A preferred embodiment is a toroidal or cylindrical core shape with interior diameter sufficiently large to place the patient through the interior, as well as other components of the device such as patient-handling hardware.

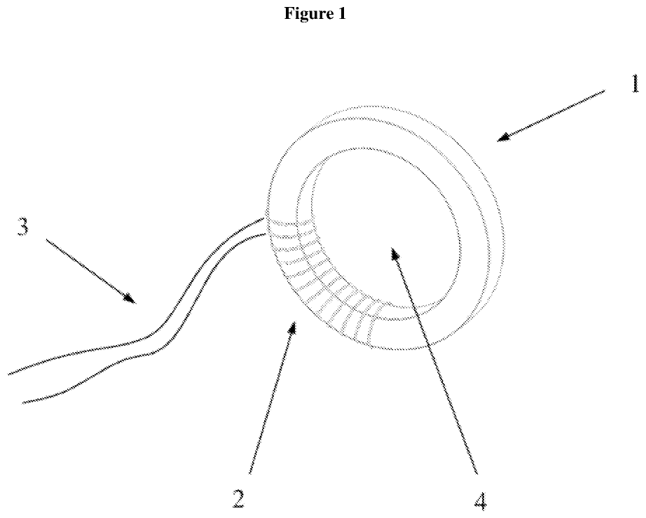

The electrical field is produced by a toroidal device shown in FIG. 1. The toroid device consists of a toroid structure (1) made of material with high magnetic permeability, such as silicon steel or other material commonly used in the cores of electrical transformers, as well as conducting windings (2) wrapped around the toroid material, supplied with electrical current by wires (3). The magnetic material of the toroid would typically have relative magnetic permeability in the range of 1,000-40,000. It is desirable for the magnetic permeability to be as large as possible. Some other materials that could be considered suitable, for example, are powdered iron, nickel-iron alloys, ferrite ceramics, and nanocrystalline alloys of iron, boron, and silicon. The conducting windings would typically be copper wire in the range of 10-28 AWG, wound into a coil arrangement on the toroid with typically 1-200 turns, depending upon other aspects of the driving circuitry.

The toroid (1) is closely wrapped with coils of current-carrying wire (2) as depicted in FIG. 1, in a manner similar to a transformer. The toroid comprises a high-magnetic-permeability magnetic material. An example of the magnetic material includes, but not limited to, silicon steel. An electrical current is ramped through these wires, which generates a strong magnetic field inside the magnetic material. This magnetic field increases or decreases as the current in the wires increases or decreases, which, through the Faraday effect, creates an associated electric field. By changing the magnitude of the magnetic field with time, an electric field is generated in the region defined by the interior region of the toroidal cores as depicted in FIG. 1.

An electrical current in the windings (2) creates a strong magnetic field inside the toroid as a result of the high magnetic permeability of the toroid material. If the current in these windings is changed in time, the changing current creates a changing magnetic field inside the toroid. This changing magnetic field, through Faraday's Law of Induction, generates an electrical field within the toroid material, but also in the region outside the toroid material, including the central region (4) of the toroid. The magnitude of the electric field generated is directly proportional to the rate of change of the magnetic field inside the toroid material.

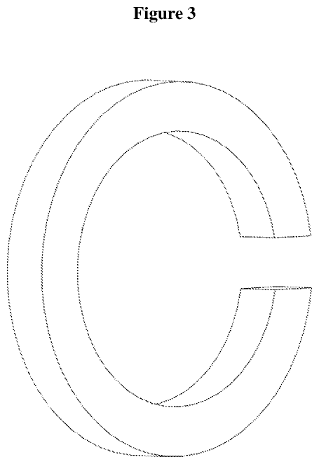

The toroid unit (1) in FIG. 1 can in principle be shaped in non-circular geometries, and doesn't have to be a completely closed structure. The circular shape and closed structure are a preferred embodiment, as they produce fields of high uniformity with low power input. With a shape that was not closed, such as a toroid with a gap, the required input power would be increased, but there would still be substantial reduction in power requirements relative to using no magnetic materials. If the electric field pulses are desired over only a small volume, the fields could be created by having a magnetic structure that is closed except for a gap wide enough to place the relevant anatomy, as in FIG. 3.

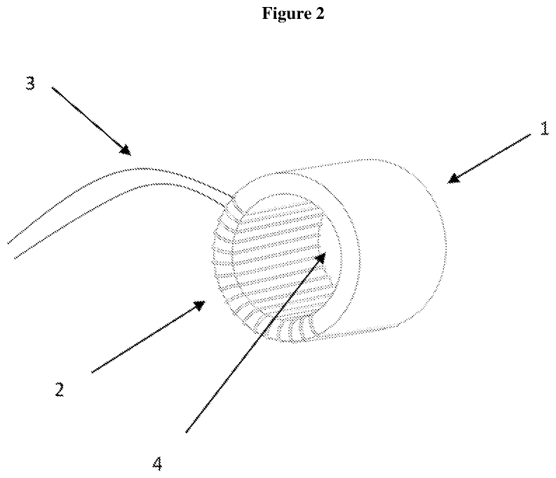

The electrical field is produced by a cylindrical device is shown in FIG. 2. The device consists of a cylinder structure (1) made of material with high magnetic permeability, such as silicon steel or other material commonly used in the cores of electrical transformers, as well as conducting windings (2) wrapped around the cylinder material, supplied with electrical current by wires (3). The magnetic material of the cylinder would typically have relative magnetic permeability above 1,000. It is desirable for the magnetic permeability to be as large as possible. Some other materials that could be considered suitable, for example, are powdered iron, nickel-iron alloys, ferrite ceramics, and nanocrystalline alloys of iron, boron, and silicon. The conducting windings would typically be copper wire in the range of 10-28 AWG, wound into a coil arrangement on the cylinder with typically 1-200 turns, depending upon other aspects of the driving circuitry.

The cylinder (1) is closely wrapped with coils of current-carrying wire (2) as depicted in FIG. 2, in a manner similar to a transformer. The cylinder comprises a high-magnetic-permeability magnetic material. An example of the magnetic material includes, but not limited to, silicon steel. An electrical current is ramped through these wires, which generates a strong magnetic field inside the magnetic material. This magnetic field increases or decreases as the current in the wires increases or decreases, which, through the Faraday effect, creates an associated electric field. By changing the magnitude of the magnetic field with time, an electric field is generated in the region defined by the interior region of the cylinder as depicted in FIG. 2.

An electrical current in the windings (2) creates a strong magnetic field inside the cylinder as a result of the high magnetic permeability of the magnetic material that constitutes the cylinder. If the current in these windings is changed in time, the changing current creates a changing magnetic field inside the cylinder. This changing magnetic field, through Faraday's Law of Induction, generates an electrical field within the magnetic cylinder material, but also in the region outside the cylinder material, including the central region (4) of the cylinder. The magnitude of the electric field generated is directly proportional to the rate of change of the magnetic field inside the cylinder material.

The cylinder unit (1) in FIG. 2 can in principle be shaped in non-circular geometries, and doesn't have to be a completely closed structure. The circular shape and closed structure are a preferred embodiment, as they produce fields of high uniformity with low power input. With a shape that was not closed, such as a cylinder with a gap, the required input power would be increased, but there would still be substantial reduction in power requirements relative to using no magnetic materials. If the electric field pulses are desired over only a small volume, the fields could be created by having a magnetic structure that is closed except for a gap wide enough to place the relevant anatomy, as in FIG. 3.

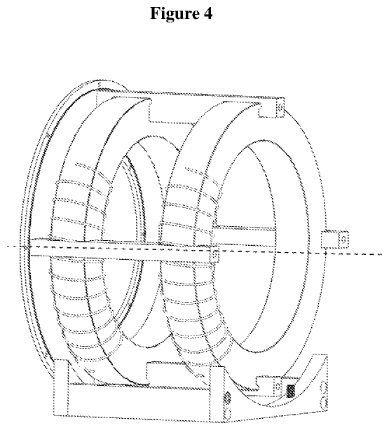

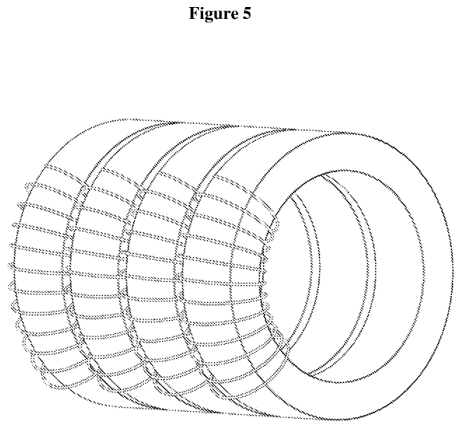

By using a multiplicity of such toroidal or cylindrical cores with carefully-designed geometrical relationships with regard to diameter and separation, large regions of high-electric-field uniformity can be generated. One embodiment is analogous to a Helmholtz Coil that is used for creating magnetic fields from electrical current. In this arrangement, as depicted in FIG. 4, the toroidal cores are aligned so they share a common axis, and separated by a distance equal to the radius of the toroidal or cylindrical cores. Another embodiment is shown in FIG. 5, in which a plurality of cylindrical cores are arranged to share a common axis. In either configuration depicted in FIGS. 4 and 5, the patient is placed along the central axis of the device. When arranged as such, the electric field runs along the axis of the toroidal or cylindrical cores, which would, in the preferred application, run along the long axis of a human patient or many types of veterinary patients. Such an arrangement maximizes the electric field uniformity for two coils. Larger regions of uniformity can be created by using more than two coils. For instance, with three coils, an arrangement analogous to a Maxwell coil for magnetic fields can be used, which involves a coil of larger radius situated halfway between two identical coils. Such designs can extend to arbitrarily large numbers of coils to increase the homogeneous volume, at the expense of system cost, weight, and complexity.

To obtain electric field pulses of a given amplitude, the coils are driven with voltage pulses. A voltage applied across the windings of the coil creates an electric field around the coil, which induces an opposing voltage in the windings, just as occurs in any inductor. The electric field that creates this opposing voltage is the desired electric field. Although some of the applied voltage goes to dissipation in the resistance of the windings and other components in the circuit, the dominant portion is responsible for creating the desired electric field. There are three primary benefits to generating the electric field with this method. First, the electric field has high spatial uniformity. Second, the electric field points tangentially to the surface of a patient lying in the device. Third, the power requirements and heat generation are very low relative to some other methods. High uniformity is desirable so that the therapy is applied in a manner consistent throughout the body or region of treatment. The usable therapeutic region for this application is considered to be where the field strength variation is less than approximately 10% in empty space.

The desirability of the electric field pointing tangentially to the surface of the patient is to minimize the reduction in electric field that occurs from polarizing water molecules inside the body. Water has a very strong polarizability (electric susceptibility), which leads to a large reduction in field inside the body. This effect is maximum for fields that point perpendicular to the surface, with reductions in electric field as high as a factor of 75-80. For electric fields pointing along the surface of the patient, the reduction can be far smaller, ranging from almost no reduction to a reduction by a factor of approximately 20.

The power requirements for this technique are very low because of the use of materials with high magnetic susceptibility in the toroidal or cylindrical cores. The power required and heat generated can be more than 10,000 times higher without the use of magnetic materials, leading to onerous requirements on electrical facilities, engineering challenges relating to heat removal, patient safety issues, and heavy special requirements for building HVAC systems.

Such power estimates can be made by considering the current densities needed to produce the desired magnetic fields, using a cylindrical geometry. One useful reference geometry to use for such a comparison is for the currents to occupy a cylindrical annulus in space from radius R1 to radius R2, over a length L, flowing primarily in an azimuthal direction. One skilled in the art can easily calculate the current density required to create a given magnetic field directed along the axis of the solenoid, and using the conductivity of copper, estimate the heat density generated. For example, using solid copper filling a cylindrical annulus with inner diameter 70 cm and outer diameter 110 cm, 1 meter long, would lead to peak heat generation higher than 40 kilowatts and require more than 2000 pounds of copper.

The peak power required in the device described in this disclosure to generate similar electric field is in the 5-20 Watt range when using high-magnetic-permeability material such as silicon steel in the toroid and is typically less than 100 Watts when using high-magnetic-permeability material such as silicon steel in the cylinder. This large reduction in power is a result of the very high magnetic permeability of the magnetic material, resulting in a magnetic field inside the magnetic material that roughly four orders of magnitude larger than it would be in empty space for the same winding pattern.

The electric field amplitude can be controlled in an ‘open-loop’ arrangement, in which the expected electric field output is known from the input voltage, the currents created, and the system resistances, or in a ‘closed-loop’ arrangement in which a feedback loop is used. The electric field can be measured in a manner described below, and that information fed back into the electronics system in a feedback loop which adjusts the applied voltage to create the desired electric field amplitude.

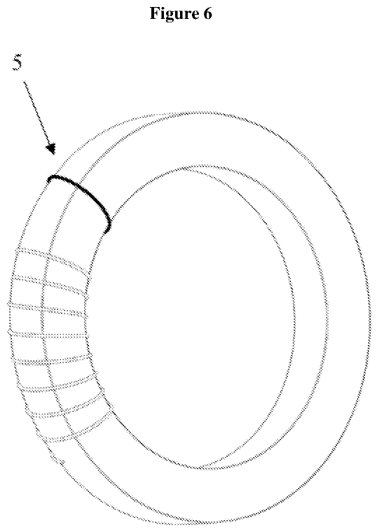

The measurement of the electric field is done through a combination of computation and measurement. Measuring a local electric field can be very difficult, but an integrated measurement can be performed around a closed loop. A loop of wire (5) that encloses the cross-section of the toroid or cylinder, as in FIG. 6 will have induced in it a voltage which is equal in magnitude to the rate of change of magnetic flux through the toroid/cylinder cross section, since nearly all of the magnetic flux is contained within the magnetic material. This integrated voltage will depend only upon how many times the wire loops around the cross section, and will otherwise be independent of the path that loop follows.

The spatial dependence of the electric field can be calculated from the known geometry and related to the voltage measured around a single loop. To perform the measurement of the voltage around a loop, a loop of wire is passed around the cross section of the toroid or cylinder and closed at a high-impedance terminal, such as the inputs to an oscilloscope or any high-input-impedance terminal. As long as the input impedance of the terminal is much larger than the resistance of the wire, then nearly the entire voltage induced around the loop will be dropped across the high-impedance terminal. This voltage around the loop can be used along with the computed spatial distribution of electric fields to obtain a local value for the electric field strength. In this capacity, the voltage induced around a single loop acts as a scaling or calibration factor to the spatial distribution.

Thus, an additional element of the device is a loop of wire (5) that can encircle the toroid or cylinder cross section once, or more than once for a higher sensitivity to the electric field. This wire is connected back to the system electronics to be used for monitoring of the electric field, or for use in the feedback loop described earlier.

The voltage pulses in the driving electronics can be created with many different types of amplifier configurations. Since it is usually desirable to have voltages driving the toroid or cylinder windings in the range of 15-100 Volts, a Class D amplifier configuration is desirable to avoid large heat dissipation in the output transistors of the amplifier. This configuration uses Pulse Width Modulation (PWM) to control the output of the amplifier and is known for its high efficiency and low cost.

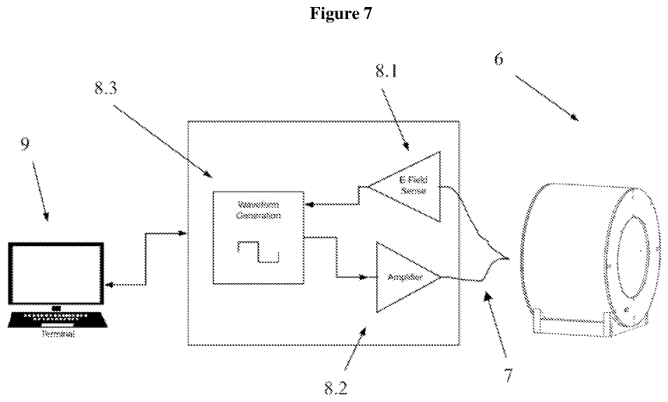

The device creating the electric field can further be incorporated into a system that can be applied in a therapeutic capacity that, when combined with pharmacological agents, can treat some types of cancers, as depicted in FIG. 7. Specifically, the system comprises one or more toroidal or cylindrical cores in an enclosure and connected to a control system for application of therapy involving electric fields. FIG. 7 shows the block diagram of the system. The toroid or cylinder device (6) produces electric field pulses on the interior region, where a patient is placed. Cables (7) connect the toroid or cylinder device to driving and sensing circuitry (8.1-8.3) that provide voltage or current pulses to the windings on the toroid unit. Sensing coils inside the toroid or cylinder unit measure the electric field produced inside the toroid or cylinder unit and can be used to control the output. A microprocessor (9) presents a user interface to the operator of the device, and interfaces to the driving and sensing circuitry to control the amplitude, duration, and spacing of the pulse, as well as to start and stop the pulses.

The driving electronics are connected to a computer that hosts a user interface that enables the user to control the pulse amplitude, duration, and spacing, as well as starting and stopping the pulse therapy. The computer can communicate with the driving electronics through a serial bus, though other choices are possible.

The pulsed electric field system can be applied in a therapeutic technique called Targeted Osmotic Lysis (TOL). See U.S. Pat. No. 8,921,320. The principle behind the technique is that the electric field pulses stimulate sodium channels in the cell membrane to open, passing more sodium into the cell. Cancer cells are known to have far more sodium channels than non-cancer cells. An increase in sodium concentration inside the cell results. A pharmacological agent blocks the exit of the sodium from the cell. The result is an increase in osmotic pressure inside the cell, causing the cell to rupture. Because cancer cells have far more sodium channels than non-cancer cells, the normal tissue is spared.

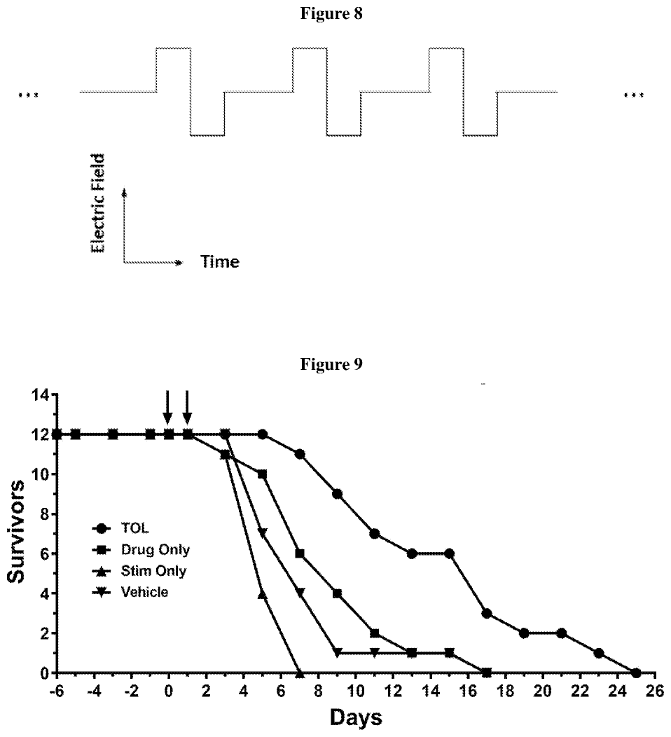

FIG. 8 shows a typical pulse train associated with the TOL application. The electric field amplitude falls in the range of 0.1 V/m to 100 V/m in free space. The pulses consist of a forward polarization of approximately 1-50 milliseconds, followed by a reverse polarization of similar duration and amplitude. The pulses are separated by 5-50 milliseconds from finish to start. The precise details of timing, duration, and amplitude may vary widely in the application.

FIG. 9 depicts in vivo validation of the therapeutic efficacy of pulsed magnetic fields inducing osmotic lysis in a breast cancer mouse model. Four groups of female, immune competent BALBc mice (n=8) with xenografts (0.7-1.2 cm diameter lower back) were established after injection of 500K highly malignant mouse breast cancer 4T1 cells in 7 mg/kg digoxin or saline (s.c. back of neck) five times at 1 hr intervals. This protocol establishes steady-state pharmacokinetics in even poorly vascularized tissues. The mice were exposed to the pulsed magnetic fields generated by a toroid device, for 30 min starting 15 min after the last injection. This treatment was administered on day 0 (first day of treatment), and on day 1. Mice were monitored for tumor growth and were sacrificed when they met the NIH criteria for humane endpoint euthanasia. As shown in FIG. 9, mice treated with TOL reached humane endpoint euthanasia criteria significantly longer than mice in the control groups.

The electric fields produced by the toroid or cylinder device may also have other therapeutic or industrial applications.

It is to be understood that the above described embodiments are merely illustrative of numerous and varied other embodiments which may constitute applications of the principles of the invention. Such other embodiments may be readily devised by those skilled in the art without departing from the spirit or scope of this invention and it is our intent they be deemed within the scope of our invention.

### LIST OF REFERENCE SIGNS

1 Toroid/cylinder structure
2 Conducting winding
3 Wire
4 The central region of the toroid structure
5 A loop of wire encircling the cross section of the toroid structure
6 Toroid/cylinder device
7 Cable
8 A driving and sensing circuitry
9 Microprocessor

1. A device for generating pulsed electric fields, comprising:
one or more toroid or cylinder structure(s) (1),
a plurality of conducting windings (2) wrapped around each toroid or cylinder structure, and
a plurality of wires (3) that supply electrical current to the conducting windings (2).

2. The device of claim 1, wherein the toroid or cylinder structure (1) is made of a magnetic material with high relative magnetic permeability

3. The device of claim 2, wherein the relative magnetic permeability of the magnetic material is 1,000 or higher.

4. The device of claim 2, wherein the magnetic material is selected from the group consisting of silicon steel, powdered iron, nickel-iron alloys, ferrite ceramics, nanocrystalline alloys of iron, boron, and silicon.

5. The device of claim 1, wherein the conducting winding (2) is made of a material selected from the group consisting of aluminum, silver, tin, galvanized steel, phosphor bronze, lead, or gold.

6. The device of claim 1, wherein the conducting winding (2) is made of copper wire in the range of 10-28 AWG.

7. The device of claim 1, wherein the conducting winding (2) is wound into a coil arrangement on the toroid or cylinder structure with 1 to 200 turns.

8. The device of claim 1, wherein a magnetic field inside the magnetic material is generated when an electrical current is ramped through the conducting windings (2).

9. The device of claim 8, wherein the magnetic field increases or decreases as the current in the conducting winding (2) increases or decreases to create an electric field.

10. The device of claim 9, wherein the electric field is generated in the interior region of the toroid or cylinder structure (1) by changing the magnitude of the magnetic field with time.

11. The device of claim 10, wherein the magnitude of the electric field generated is directly proportional to the rate of change of the magnetic field inside the magnetic material of the toroid or cylinder structure.

12. The device of claim 1, wherein the toroid or cylinder structure (1) is in a circular shape.

13. The device of claim 1, wherein the toroid or cylinder structure (1) is in a non-circular shape.

14. The device of claim 1, wherein the toroid or cylinder structure (1) is a closed structure.

15. The device of claim 1, wherein the toroid or cylinder structure (1) is a non-closed structure.

16. The device of claim 1, wherein at least two toroid or cylinder structures (1) are arranged coaxially.

17. The device of claim 16, wherein the at least two toroid or cylinder structures (1) are separated by a distance equal to the radius of the toroid or cylinder structure.

18. The device of claim 1, wherein a human or animal subject is placed along the central axis of the device.

19. The device of claim 1, wherein the electric field pulses of a given amplitude is created by a voltage applied across the conducting windings (2) of the toroid or cylinder structure (1).

20. The device of claim 1, wherein the amplitude of the pulsed electric field is controlled in an ‘open-loop’ arrangement, in which the expected electric field output is known from input voltage, currents created, and system resistances.

21. The device of claim 1, further comprising an additional coil winding used for sensing the electric field generated within the device.

22. The device of claim 21, wherein the additional coil winding comprises a loop of wire (5) encircling the cross section of the toroid or cylinder structure at least one turn for a higher sensitivity to the electric field.

23. The device of claim 22, wherein the loop of wire (5) induces a voltage which is equal in magnitude to the rate of change of magnetic flux through the cross section of the toroid or cylinder structure.

24. The device of claim 22, wherein the loop of wire (5) is closed at a high-impedance terminal for the measurement of the voltage around the loop.

25. A system for therapeutic treatments involving electric fields, comprising:
a toroid or cylinder device (6) for generating pulsed electric fields,
a driving and sensing circuitry (8),
a plurality of cables (7) connecting the device (6) to the driving and sensing circuitry (8), and
a microprocessor (9) providing a user interface for operating the device (6) and the driving and sensing circuitry (8).

26. The system of claim 25, wherein the toroid or cylinder device (6) comprises one or more toroid or cylinder structure(s) (1), conducting windings (2) wrapped around the toroid or cylinder structure, and wires (3) that supply electrical current to the conducting windings (2).

27. The system of claim 25, wherein the pulsed electric fields are used for treating cancers when combined with pharmacological agents.

28. The system of claim 25, wherein the pulsed electric fields are used in targeted osmotic lysis for cancer treatment when combined with pharmacological agents.

29. The system of claim 28, wherein the amplitude of the electric field falls in the range of 0.1 V/m to 100 V/m in free space.

30. The system of claim 28, wherein the pulses of electric field consist of a forward polarization of approximately 1-50 milliseconds, followed by a reverse polarization of similar duration and amplitude.

31. The system of claim 28, wherein the pulses of the electric field are separated by 5-50 milliseconds from finish to start.

32. A method for therapeutic treatments involving electric fields, comprising generating pulsed electric fields by a toroid or cylinder device (6), wherein the toroid or cylinder device (6) comprises one or more toroid or cylinder structure(s) (1), conducting windings (2) wrapped around the toroid or cylinder structure, and wires (3) that supply electrical current to the conducting windings (2).

33. The method of claim 32, further comprising using pharmacological agents for in targeted osmotic lysis.
